(19) United States
(12) Patent Application Publication
(10) Pub. No.: US 2014/0329334 A1
(43) Pub. Date: Nov. 6, 2014

(54) METHOD FOR MONITORING, DIAGNOSIS AND/OR PROGNOSIS OF ACUTE KIDNEY INJURY IN EARLY STAGE

(71) Applicant: Carlos Ernesto Irarrazbal Muñoz, Santiago (CL)

(72) Inventor: Carlos Ernesto Irarrazbal Muñoz, Santiago (CL)

(73) Assignee: Universidad de losAndes, Santiago (CL)

(21) Appl. No.: 14/346,679

(22) PCT Filed: Sep. 22, 2011
(86) PCT No.: PCT/IB2011/054187

§ 371 Date: Jul. 21, 2014

(51) Int. Cl. G01N 33/543

(52) U.S. Cl. CPC G01N 33/543; G01N 2333/46; G01N 2800/347
USPC 436/501

(57) ABSTRACT

The present invention relates to a method and a kit for monitoring, diagnosis, prognosis of acute kidney injury in early stage and determination of treatment in subjects suffering thereof. The method comprises the steps of a) providing a urine sample; b) enriching the urine sample in exosomes present in the urine sample using at least one step of immunopurification; c) detecting an acute kidney injury (AKI) marker in the exosome. The invention also relates to a kit for determining the presence and/or level of a specific kidney injury marker, for simple and early determination of the onset of AKI in a subject, the kit comprising means for enriching the urine sample in exosomes using at least one step of immunopurification, and means for detecting a predetermined kidney injury marker of a condition.

The method and kit of the invention have the advantage to provide a specific, simple and early detection of kidney injury markers that is no possible with the standard methods applied until now. The method and kit of the invention also provide the advantage of identifying the specific structure that is injured in the kidney, since it uses specific antibodies for specific proteins present in specific structures of the kidney.

### FIELD OF THE INVENTION

The present invention relates to a method and a kit for monitoring, diagnosis and/or prognosis of acute kidney injury in early stage and determination of treatment in subjects suffering thereof.

### BACKGROUND OF THE INVENTION

The kidney is an organ that serves multiple functions in the body, among which stand the elimination of body waste substances that are produced during metabolism, and return to the blood of substances that are necessary for the body, so as well as regulating the volume and composition of body fluids. The hydroelectrolytic balance of the body is maintained due to the kidney function (Guyton A., Hall J. E. 2001, Medical Physiology Treaty, tenth edition. Mexico DF, Mexico: McGraw-Hill Interamericana).

Kidney diseases are one of the most important causes of death in many countries. As early as 1994, more than 15 million of people presented kidney diseases in the United States, causing deterioration in life quality of the patient and death (Guyton A., Hall J. E. 2001, Medical Physiology Treaty, tenth edition. Mexico DF, Mexico: McGraw-Hill Interamericana).

The clinical manifestations of kidney disease can be grouped into fairly well defined syndromes. Some are specific to glomerular diseases, and others are present in diseases that affect any of the renal structures. These diseases are among the causes of major morbidity and death in many countries (Guyton A., Hall J. E. 2001, Medical Physiology Treaty, tenth edition. Mexico DF, Mexico: McGraw-Hill Interamericana).

Severe kidney disease can be grouped into two main categories:

Pathologies

Kidney failure is a clinical condition in which the kidneys fail to function properly, reducing the glomerular filtration rate. Clinically, this fault is divided into two groups: acute kidney injury (AKI) and chronic renal failure (CRF) (Guyton A., Hall J. E. 2001, Medical Physiology Treaty, tenth edition. Mexico DF, Mexico: McGraw-Hill Interamericana).

Renal Failure

Acute Kidney Injury

Among its symptoms are included oliguria or anuria (decrease or absence of urinary excretion), with azotemia, which corresponds to the accumulation of nitrogenated products in the blood. Metabolic waste products are also accumulated due to water retention, which determines an overload of fluid and salts, which results in edema and hypertension.

One of the biggest threats of the symptoms is the potassium retention (hyperkalemia), above 8 mEq/L, which could be fatal. Kidneys do not excrete a normal amount of hydrogen ions, resulting in a manifestation of metabolic acidosis.

If the illness continues without resolution, it can reach a complete anuria, which can lead to death within 8 to 14 days (Guyton A., Hall J. E. 2001, Medical Physiology Treaty, tenth edition. Mexico DF, Mexico: McGraw-Hill Interamericana).

The AKI is divided into 3 main categories: pre-renal, intra-renal and post-renal.

TABLE 1
Causes of acute kidney injury (Guyton A., Hall J.
E. 2001, Medical Physiology Treaty, tenth edition.
Mexico DF, Mexico: McGraw-Hill Interamericana).
Some causes of pre-renal acute kidney injury

| | |
|---|---|
| Intravascular volume depletion | Hemorrhage (traumatic, surgical, |
| | puerperal, digestive) |
| | Diarrhea |
| | Burns |

Some causes of pre-renal acute kidney injury

| | |
|---|---|
| Primary renal hemodynamic | Renal artery stenosis |
| disorders | Embolism or thrombosis of the renal |
| | artery or vein |
| | Excessive blocking of prostaglandin |
| | synthesis (aspirin) |
| Peripheral vasodilatation and | Anaphylactic shock, sepsis, serious |
| resulting hypotension | infections |
| | Anesthesia |

Some causes of intra-renal acute kidney injury

| | |
|---|---|
| Injuries to small vessels, | Vasculitis (polyarteritis nodosa) |
| glomeruli, or both | Cholesterol emboli |
| | Malignant hypertension |
| | Acute glomerulonephritis |
| Tubular epithelial injury | Ischemic acute tubular necrosis |
| (tubular necrosis) | Acute tubular necrosis by toxins |
| | (heavy metals, ethylene glycol, |
| | insecticides, poisonous mushrooms, |
| | carbon tetrachloride) |
| Injury of the renal inter- | Acute pyelonephritis |
| stitium | Acute allergic interstitial nephritis |

Some causes of post renal acute kidney injury

| | |
|---|---|
| Bilateral obstruction of the | Kidney stones |
| ureters or renal pelvis | Hematic clots |
| Bladder obstruction | |
| Obstruction of the ureters. | |

In industrialized countries, AKI is a pathological condition mainly acquired in hospitals and originated by several factors, such as sepsis, surgical interventions, particularly cardiac surgeries, ischemia, administration of nephrotoxins, therefore, there is a need of modern diagnosis techniques and treatments for preventing and diminish the impact of AKI (Vukusich A., Alvear F., Villanueva P., Gonzalez C., Olivari F., Alvarado N., Zehnder C. 2004, Rev. Med. Chile, 132:1355-1361).

Most of the patients that develop an AKI episode recover acceptable levels of kidney function, so they do not depends on dialysis, although 10 to 20% of these patients finally need permanently dialysis (Fauci, A. 2009, Harrison, Principles of intern medicine. Mexico, F.D. Interamericana—McGraw Hill). Between 5 to 7% of the hospitalized patients have complications relating to an AKI episode, causing 30% of the costs in ICU (Fauci, A. 2009, Harrison, Principles of intern medicine. Mexico, F.D. Interamericana—McGraw Hill). AKI is a severe problem, therefore, there are a lot of efforts to develop an early intervention of the pathology, especially in high risk patients, such as ICU patients (Vukusich A., Alvear F., Villanueva P., Gonzalez C., Olivari F., Alvarado N., Zehnder C. 2004, Rev. Med. Chile, 132:1355-1361; Schrier R. 2010, Nat. Rev. Nephrol, 6:56-59).

Chronic Renal Failure

As earlier mentioned, chronic renal failure is a consequence of the irreversible loss of a large number of functioning nephrons.

Kidney efficiency allows us to maintain relatively normal blood concentrations of most electrolytes and an appropriate volume of body fluids, while the number of functioning nephrons remains above the 20 to 30% of normal. No serious clinical symptoms are present above this figure (Guyton A., Hall J. E. 2001, Medical Physiology Treaty, tenth edition. Mexico DF, Mexico: McGraw-Hill Interamericana).

Among the most important causes of chronic renal failure are those described in Table 2.

TABLE 2
Causes of chronic renal insufficiency (Guyton A., Hall
J. E. 2001, Medical Physiology Treaty, tenth edition.
Mexico DF, Mexico: McGraw-Hill Interamericana).

| | |
|---|---|
| Metabolic disorders | Diabetes mellitus |
| | Amyloidosis |
| Renal vascular disorders | Atherosclerosis |
| | Nephrosclerosis-hypertension |
| Immune Disorders | Glomerulonephritis |
| | Polyarteritis nodosa |
| | Lupus erythematosus |
| Infections | Pyelonephritis |
| | Tuberculosis |
| Primary tubular disorders | Nephrotoxins (analgesics, heavy metals) |
| Urinary tract obstruction | Kidney stones |
| | Enlarged Prostate |
| | Urethral stricture |
| Congenital disorders | Polycystic disease |
| | Congenital absence of kidney tissue |
| | (renal hypoplasia) |

End Stage Renal Disease (ESRD)

In many cases, the initial lesion of the kidney leads to progressive deterioration of renal function, and a continuing loss of nephrons, to the point that the patient must enter a program for dialysis or a kidney transplant to survive. This condition is called chronic renal failure (Kumar V., Cotran R. 2000, Structural and Functional Pathology of Robbins, sixth edition, Madrid, Spain: Elsevier).

TABLE 3
Most frequent causes of ESRD (Kumar V., Cotran R.
2000, Structural and Functional Pathology of Robbins,
sixth edition, Madrid, Spain: Elsevier).

| Cause | Percentage of total patients with ESRD (%) |
|---|---|
| Diabetes Mellitus | 41 |
| Hypertension | 28 |
| Glomerulonephritis | 11 |
| Polycystic kidney disease | 3 |
| Other/Unknown | 18 |

Kidney Transplant

Kidney transplant is the most effective treatment for ESRD. The success of transplant depends on many factors such as type of donor. The donor from which comes the kidney transplant can be either dead or alive. All transplanted organs are submitted to an acute injury due to ischemia and reperfusion process associated with the transplant.

Living donor transplant is an excellent therapeutic option for the treatment of end stage chronic renal failure, especially in young patients. It allows following a defined pre-transplant protocol to help the better survival of the organ, together with a small ischemic time (less than 30 minutes). Survival becomes greater than 10 years, and higher in 17 to 20% compared to transplants with organs from cadaveric donors.

In the case of cadaveric donor transplant, the patient enters a waiting list and the selection is made regarding the degree of ABO and HLA compatibility. Among the selection criteria are the age, similarity between the body mass index of donor and recipient, if it corresponds to the first transplant, and if the transplant is of one kidney or both. In this case the organ ischemic time could increase several hours (even 1 or 2 days), which increases the deterioration of transplanted organ. There are also further complications, as for chronic dialysis patients.

The renal transplant patient requires careful monitoring, initially in intensive care unit (ICU) or transplant unit and later, hospitalized or as outpatient. Several surgical or medical complications may occur during monitoring. Although some complications are premature and could be treated while still confined in the ICU or transplant unit, this monitoring should be extended throughout the whole life of the patient, as there are complications that can arise even after a long period of time after receiving transplant (late complications). During the graft function, it is necessary to administer immunosuppressive therapy to increase survival of the transplanted organ, survival of the patient, and to improve his/her quality of life. This treatment is adjusted to the clinical and serological condition of each patient. Along with immunosuppressants, other aspects should take into account within the long-term monitoring of renal transplant, which include encouraging the patient to follow a healthy lifestyle, monitoring of proteinuria and creatinine in 24 hours urine, the administration of treatment for hyperlipidemia, blood pressure control, the possible occurrence of DE NOVO diabetes, and in the case of graft dysfunction, the possible occurrence of chronic renal failure.

Patients with renal transplant are a good model of AKI in humans. These patients have the advantage that there are no other pathologies that may alter the studied biomarkers. However, they have the disadvantage that are under immunosuppressive pharmacology.

Diagnostic of Acute Kidney Injury (AKI)

Acute Kidney Injury (AKI) can be classified in different levels depending on the stage corresponding to the deterioration of renal function.

AKI is classified depending on a variety of parameters, the most common are determination of serum creatinine (SCR) and diuresis (D), which appear late in the development of the disease, and as such, do not allow AKI diagnostic in early stages.

Creatinine

Creatinine is a metabolic byproduct formed in muscle tissue by degradation of phospho-creatine, which is eliminated from the body through filtration at glomerular level. Total rate of creatinine production will depend on muscular mass, muscular activity, sex, age, and total consumption of proteins. These variables also affect creatinine plasma levels. In spite of these limitations, the most used method for diagnostic of AKI is when serum creatinine levels are above 0.6-1.2 mg/dl.

Creatinine Clearence

Creatinine clearance is the amount of creatinine which is eliminated from the blood flow in a period of time. This parameter is determined to estimate the glomerular filtration. Usual levels of creatinine clearance are between 125-150 ml/min and slightly lower in women.

Oliguria

Oliguria is defined as a decrease in urine production, with a value below 400 ml per day, considering 400 ml as the minimal amount of urine that should be released in a normal metabolic state to eliminate daily produced solutes.

Uremia

Uremia corresponds to elevated levels of urea in blood. Determination of this parameter is through determination of blood urea nitrogen (BUN), which in normal values is between 8 to 18 mg/dl.

Risk Injury Failure Loss End-Stage Kidney Disease (RIFLE) Classification

RIFLE is a tool proposed to classify different stages of AKI: Risk, Injury, Failure, Loss, End-stage kidney disease. It is based on serum creatinine (SCR) and diuresis (D) levels of the patient. (Carrillo R., Castro J. “009. RIFLE scale. Journal of Mexican association of critic medicine and intensive therapy, 23(4):241-244). As used in the present invention, AKI 1, AKI 2, and AKI 3, corresponds to stages R, I and L of RIFLE respectively.

Neutrophil Gelatinase-Associated Lipocalin (NGAL)

Using genomics and proteomics, a series of molecules as potential markers of acute kidney injury have been identified, including NGAL, Cystatin-3, KIM-1, IL-1beta, and IL-18.

NGAL is usually expressed in low concentrations, and grown significantly for epithelial damage (Schmidt-Ott K. M., Mori K., Kalandadze A., Li J. Y., Paragas N., Nicholas T., Devarajan P., Barasch J. 2006, Curr Opin Nephrol Hypertens, 15:442-449; Cowland J. B., Borregaard N. 1997, Genomics, 45:17-23).

NGAL is a small protein that belongs to the lipocalin family. The human NGAL is a single polypeptidic chain, with disulfide bridges, with 178 amino acidic residues, with a mass of 23 KDa, and its dimeric form of 46 KDa (Kjeldsen L., Johnsen A. H., Sengelov H., Borregaard N. 1993, J Biol Chem, 268:10425-10432.). This protein is expressed in neutrophils of certain epithelia, including the epithelium of proximal renal tubules. NGAL is a secreted protein and is characterized by its ability to bind to small and hydrophobic molecules in its form of structurally conserved pocket, through β-pleated sheets to form macromolecular complexes (Uttenthal L. O. 2005 Clin Lab, 29:39-41).

As already mentioned, it is very difficult to detect kidney damage in early stage. NGAL has partially overcome the general obstacles on this issue, and has proven been useful in the diagnosis of acute kidney injury, showing that it is possible to make this diagnosis at early stage (Zappitelli M., Washburn K. K., Arikan A. A., Loftis L., Ma Q., Devarajan P., Parikh C. R., Goldstein S. L. 2007 Crit Care, 11: R84.).

Numerous studies show that NGAL has a significant increase in patients with acute kidney injury, but not in the corresponding controls, this increase been produced within the first 24 to 48 hours, and prior to the increase in creatinine. This marker is used in both plasma and urine, but it still requires a complete evaluation in different clinical areas (Mishra J., Dent C., Tarabishi R., Mitsnefes M. M., Ma Q., Kelly C., Ruff S. M., Zahedi K., Shao M., Bean J., Mori K., Barasch J., Devarajan P. 2005, Lancet, 365:1231-1238; Devarajan P. 2007, Contrib Nephrol, 156:203-212).

It has also been shown an increase in this marker in urine after surgeries such as cardiopulmonary bypass in adults (Wagener G., Jan M., Kim M., Mori K., Barasch J. M., Sladen R. N., Lee H. T. 2006, Anesthesiology, 105:485-491), percutaneous coronary interventions (Bachorzewska-Gajewska H., Malyszko J., Sitniewska E., Malyszko J. S., Dobrzycki S. 2006, Am J Nephrol, 26:287-292), coronary angiography (Bachorzewska-Gajewska H., Malyszko J., Sitniewska E., Malyszko J. S., Dobrzycki S. 2007, Nephrol Dial Transplant, 22:295-296.), and also cardiac procedures in children (coronary bypass in children) (Mishra J., Dent C., Tarabishi R., Mitsnefes M. M., Ma Q., Kelly C., Ruff S. M., Zahedi K., Shao M., Bean J., Mori K., Barasch J., Devarajan P. 2005, Lancet, 365:1231-1238).

The limitation of measuring NGAL in urine and plasma as a marker of AKI, stating the current stage of investigations, is that NGAL is still being evaluated as adequate renal marker. Additionally, it is declared that if NGAL is exclusively of renal origin, this biomarker would be one of the best markers of kidney damage of tubule cells that could be conceived. In urine, NGAL shows an increase of 10,000 times the concentration in the more serious kidney damage above normal levels. In plasma the maximum increase is about 100 times. This makes NGAL been potentially a very sensitive marker of different degrees of renal injury. However, the lower end of this wide dynamic range is occupied by the increases of NGAL due to extra-renal sources in diseases such as cancer and other (www.clionline.com). Our innovative approach seeks to address this question by means of the analysis of NGAL in exosomes from the kidney, particularly, specific exosomes from specific kidney structures to establish early and noninvasive diagnosis of AKI.

Aquaporin 1 (AQP1)

Aquaporin 1 (AQP1) is an integral membrane protein and was the first of its kind to be structurally and functionally characterized from human red blood cells. The protein has a tetramer structure, and each of its subunits has functionality by itself (Preston G. M., Jung J. S., Guggino W. B., Agre P. 1993, J Biol Chem, 268:17-20.). It has a weight of 28 KDa (Friedman M. 2008, Principles and models of biological transport, second edition, New York, USA: Springer), and its pattern of expression depends on the age and the tissue being examined (kidney, lung, brain and eyes) (Bondy C., Chin E., Smith B. L., Preston G. M., Agre P. 1993, Proc Natl Acad Sci USA, 90:4500-4504).

This channel is strongly expressed in the renal proximal tubule, in the epithelium of Henle's loop in its descending portion and in the endothelium of the vasa recta. The high concentration of this protein in the descending loop of Henle (25% of total protein), suggests an essential role in the renal concentrating mechanism. This has been demonstrated in studies that have generated transgenic mice in which expression for AQP1 gene has been knocked out, resulting in decrease of water permeability in the proximal tubule, so that they can not concentrate their urine (Ma T., Yang B., Gillespie A., Carlson E. J., Epstein C. J., Verkman A. S. 1998, J Biol Chem, 273:4296-4299). The abnormal phenotype associated with deletion of AQP1 raises the possibility that other mammalian aquaporins have an important physiological function, since in a report of 3 apparently normal non-AQP1 patients, the analysis of fluid intake or physiological response to some stress as dehydration was not included (Preston G. M., Smith B. L., Zeidel M. L., Moulds J. J., Agre P. 1994, Science, 265:1585-1587).

Aquaporin 2 (AQP2)

Aquaporin 2 (AQP2) is an integral membrane protein that serves as a channel for water passage. This channel is regulated by vasopressin, and is located in the connecting tubule and collecting duct of the kidney, in its apical region (Fushimi K., Uchida S., Hara Y., Hirata Y., Marumo F., Sasaki S. 1993, Nature, 361:549-552).

Studies with transgenic mice have been conducted because of the importance of this protein. Mice have been modified to selectively express this aquaporin in the connecting tubule and not in the collecting duct. Also mice completely deficient in this protein have been developed. It has been observed that deficient mice die postnatally (5-12 days), while mice, which only the expression in the collecting duct was blocked, grow to adulthood, showing decreased body weight, 10 times of increase in urine production and decreased urine osmolarity. When deprived of water for 3 hours, there is no significant change in urine osmolarity, demonstrating that there are not compensatory mechanisms (Rojek A., Füchtbauer E. M., Kwon T. H., Frøkiaer J., Nielsen S. 2006, Proc Natl Acad Sci USA, 103:6037-6042).

In the absence of vasopressin, hypertonicity induces an accumulation of AQP2 in the plasma membrane within 10 minutes. This occurs in the main cells of the collecting duct of rat kidney IN SITU, and also in several kidney epithelial lines, demonstrating its importance in cases of stress to the kidney (Hasler U., Nunes P., Bouley R., Lu H. A., Matsuzaki T., Brown D. 2008, J Biol Chem, 283:26643-26661).

It is also known that there are many recessive mutations of the gene expressing the protein (Leduc-Nadeau A., Lussier Y., Arthus M. F., Lonergan M., Martinez-Aguayo A., Riveira-Munoz E., Devuyst O., Bissonnette P., Bichet D G. 2010, J Physiol, 588:2205-2218), and also dominant mutation (Mulders S. M., Bichet D. G., Rijss J. P., Kamsteeg E. J., Arthus M. F., Lonergan M., Fujiwara M., Morgan K., Leijendekker R., Van der Sluijs P., Van Os C. H., Deen P. M. 1998, J Clin Invest, 102:57-66.) that can produce nephrogenic diabetes insipidus.

Aquaporin 3 (AQP3)

Aquaporin 3 (AQP3) is an integral membrane protein, with a weight of 30 kDa (Wakayama Y., Jimi T., Inoue M., Kojima H., Shibuya S., Murahashi M., Hara H., Oniki H. 2002, Histochem J, 34:331-337) and is expressed in the basolateral membrane of epithelial cells of renal collecting duct. Unlike other aquaporins, this protein can also transport glycerol (Ma T., Frigeri A., Hasegawa H., Verkman A. S. 1994, J Biol Chem, 269:21845-21849.).

In experiments where expression for AQP3 gene is knocked out in mice, polyuria, decreased expression of AQP2, particularly in the renal cortex, and decreased urine osmolarity is observed. The liability of AQP3 in these changes was later proven knocking the expression of other aquaporins (Ma T., Song Y., Yang B., Gillespie A., Carlson E. J., Epstein C. J., Verkman A. S. 2000, Proc Natl Acad Sci USA, 97:4386-4391).

Other proteins that are preferably and differentially expressed in specific structures of the kidney have been identified, such as for example, NKCCC2 in the Henle's loop, and NHE-3 and NaPiII in the proximal tubule.

Urinary Exosomes

The group of Dr. Mark Knneper has developed proteomic studies in healthy human urine. These studies have analyzed the cellular and exosomal urinary fractions, obtained by differential centrifugation (Knepper M. A., Pisitkun T., Shen R. F. 2004, Proc Natl Acad Sci USA, 101:13368-13373). The exosomal fraction has received particular interest because it is believed to be a rich source of representative proteins of the renal epithelium.

Exosomes are produced from the endocytosis of apical membrane proteins. After this, the endosome fuses with the multivesicular body (MVB). Consequently, apical membrane proteins are segregated in the MVB outer membrane and internalized by invagination of the membrane. Finally, the outer membrane of the MVB fuses with the apical membrane, releasing their internal vesicles, called exosomes, in the urinary space (Knepper M. A., Pisitkun T., Shen R. F. 2004, Proc Natl Acad Sci USA, 101:13368-13373). The proteins currently identified in urinary exosomes correspond to plasma membrane proteins (NKCC2, CD24, etc.) cytoplasm proteins (GAPDH, etc.) and nuclear proteins (AFT3 and WT-1) (Zhou H., Cheruvanky A., Hu X., Matsumoto T., Hiramatsu N., Cho M. E., Berger A., Leelahavanichkul A., Doi K., Chawla L. S., Illei G. G., Kopp J. B., Balow J. E., Austin H. A. 3rd, Yuen P. S., Star R. A. 2008, Kidney Int, 74:613-621).

State of the Art

US2010203529 describes exosomes that can be used for detecting biomarkers in diagnostic, for example the stage or progression of a disease, describing also that biomarkers from cell-of-origin can be used to further determine treatment regimes for diseases and establishing the treatment efficacy.

EP2191276 the document describes a prenatal diagnosis method by isolating exosomes from a fluid, wherein the exosomes are identified by a specific biomarker, in particular CD24.

GB2463401 describes a method for characterising phenotype, diagnosing a disease by determining a bio-signature of an exosome in a sample from the subject. The markers mentioned comprise miRNA profiles, or antigens including CD63, CD9, CD81, B7H3, EpCam, PSCA, TNFR, MFG-E8, Rab, SETAP, PCMA or 5T4. Further, the method mentions that it can be used to determine the cell of origin of the exosomes for profiling physiological states or determining phenotypes.

WO2009115561 describes a polypeptide for identification of membrane vesicles or exosomes. Furthermore, the invention describes immunogenic preparations for preventing and/or treating an infection due to a pathogen or a tumoral antigen.

KR20070058441 describes methods and compositions for use in immunosupression reactions. The compositions comprise exosomes with immunosupressive activity, wherein the exosomes can be derived from different cell types, mainly from the immune system. Furthermore, the exosomes can be exposed to molecules to enhance the immunosupressive activity. The exosomes are used in the treatment of diseases or disorders associated with immune system malfunction.

US2007254351 describes a method for isolation of hepatitis C virus, comprising separation of exosomes from blood plasma from an individual infected with the virus.

AU2004203482 describes membrane vesicles comprising molecules (exosomes), wherein the molecules are from the major histocompatibility complex, and wherein the exosomes are used as immunogen or for diagnostic purposes.

US2004197314 describes compositions and methods to express a polypeptide in the membrane of a vesicle (exosome), focused mainly in the synthetic production of exosomes.

CA2453198 describes exosomes which can be used in identification and quantification of immune suppressive factors in biological fluids of cancer patients. These exosomes can be used alone or in combination with other immunological assays as a prognosis indicator for cancer patient.

EP1523990 describes exosomes derived from tumor cells. These exosomes have tumor specific antigens and molecules for stimulation of lymphocytes.

Zhou et al (2006) describe urinary exosomes carrying renal dysfunction and structural injury markers from an acute kidney injury model in animals. The exosomes were obtained by centrifugation, and kidney injury markers were found. (Exosomal Fetuin-A identified by proteomics: a novel urinary biomarker for detecting acute kidney injury. Zhou H., Pititkun T., Apont A., Yuen P. S., Hoffert J. D., Yasuda H., Hu. X, Chawla L., Shen R-F., Knepper M. A., Star R., 2006, Kidney Int. 70(10):1847-1857).

Zhou et al (2008) describe urinary exosomes from nephron segments that are rich in kidney injury biomarkers. They describe isolation of these exosomes by differential centrifugation, and further detection of transcription factors by Western blotting. They found that these markers were detectable in the exosomes but not in the complete urine in subjects with a renal disease, but no markers were found in normal healthy individuals. (Urinary exosomal transcription factors, a new class of biomarkers for renal disease. Zhou H. Cheruvanky A., Hu X., Matsumoto T., Hiramatsu N., Cho M. E., Berger A., Leelahavanichkul A., Doi K., Chawla L. S., Illei G. G., Kopp J. B., Balow J. E., Austin H. A. 3rd, Yuen P. S., Star R. A. 2008, Kidney Int. 74:613-621).

Devarajan (2007) proposes a panel of acute kidney injury (AKI) markers for early detection of injury, as well as predicting the outcome of kidney injury in a patient. (Proteomics for biomarker discovery in acute kidney injury. Devarajan P., Williams L. M. 2007, Semin. Nephrol. 27(6):637-651).

Lock (2010) reviews renal markers which can be used as indicators of kidney damage, focused on the use of microarray technology to detect genes that are upregulated in cases of renal or kidney injury. (Sensitive and early markers of renal injury: where are we and what is the way forward? Lock E. 2010, Toxicological Sciences 1(116):1-4).

Alvarez et al (2010) described in a congress that NGAL could be used as a kidney failure marker and furthermore, it could also be used as a recovery predictor after kidney transplantation. (Pilot study for evaluating urinary exosomal fraction as kidney dysfunction biomarker in renal transplant. Alvarez s., Suazo C., Boltansky A., Urzu M., Carvajal D., Innocenti G., Vukusich A., Hurtado M., Campos D., Yen C., Villanueva S., Flores M., Marquez J., Rogello A., Irarrazabal C. E. 2010, VII Latin American Congress of Acute Kidney Injury: 29. Coquimbo-Chile).

Boltansky et al (2010) described NGAL as a potential biomarker indicator for organ recovery predictor in cases of kidney transplantation (NGAL in urinary exosomes as a source of kidney dysfunction biomarker in renal transplantation. Boltansky A., Alvarez S., Vukusich A., Hurtado M., Ursu M., Innocenti G., Carvajal D., Suazo C., Villanueva S., Carreno J., Altuzarra R., Yen C., Tapia D., Irarrazabal C. E. 2010, Renal Week, Denver, Colo., J Am Soc Nephrol 21:959).

The documents found in the art show that recent efforts have been focused in the description and characterization of transcription factors and biomarker molecules from specific exosomes from different types of cells, among which renal cells defining renal injury stand out. A series of method describing isolation of exosomes are also addressed, together with biomarkers and diagnostic procedures. Said documents describe similar research as the present invention, suggesting the use of exosomes as a pathology marker (US2010203529, GB2463401, Zhou H. ET AL 2006, Devarajan 2007, and Lock 2010) and in particular, acute renal injury (Zhou, H. ET AL 2008, Zhou H. ET AL 2006, Devarajan 2007, and Lock 2010), specifying the use of particular markers, such as for example KIM-1, NGAL, IL-8, and Cystatin. Nevertheless, the present invention shows relevant differences with the previous art. In particular, Zhou, H. ET AL 2008 does not describe the use of antibodies, such as aquaporin-1, aquaporin-2, aquaporin-3 (AQPs), NKCC2, NHE-3, and NaPiII antibodies, which would allow immunopurification of exosomes containing these molecules (AQPs, NCCK2, NHE-3, NapiII) allowing a better and more specific identification and diagnostic of renal injury. Zhou H. ET AL 2006 does not describe specific immunoprecipitations nor some exosomal markers used in the present invention. Devarajan 2007 does not describe the use of specific immunopurification of specific renal areas based on the use of anti-AQPs, anti-NKCC2, anti-NHE-3, and/or anti-NaPiII. Lock 2010 does not describe specific immunopurification.

Alvarez et al (2010) and Boltansky et al (2010) propose the use of NGAL as an indicator of kidney injury and as a predictor of organ recovery in cases of kidney transplantation, but none of them described the stage of immunopurification of exosomes in order to have a more accurate determination of a kidney injury marker, as opposed to the present invention.

Thus, the previous art does not describe sufficient background to affect the novelty or inventiveness of the present invention. The present invention is based on the immunopurification of exosomes containing AQP-1, AQP-2, AQP-3 (AQPs), NKCC2, NHE-3, and/or NaPiII.

Furthermore, the examples of application show that the procedure is accurate in determining renal injury in a patient.

### SUMMARY OF THE INVENTION

The present invention relates to a method for monitoring, diagnosis and/or prognosis of acute kidney injury in early stage and determination of treatment in subjects suffering thereof, the method comprising the steps of a) providing a urine sample; b) enriching the urine sample in exosomes present in the urine sample using at least one step of immunopurification; c) detecting an acute kidney injury (AKI) marker in the exosome.

The invention further comprises a diagnostic kit for determining the presence and/or level of a specific kidney injury marker, for simple and early determination of the onset of AKI in a subject, the kit comprising means for enriching the urine sample in exosomes, using at least one step of immunopurification, and means for detecting a predetermined kidney injury marker of a condition.

### DETAILED DESCRIPTION OF THE INVENTION

The present invention relates to a method for monitoring, diagnosis and/or prognosis of acute kidney injury in early stage and determination of treatment in subjects suffering thereof, the method comprising the steps of a) providing a urine sample; b) enriching the urine sample in exosomes present in the urine sample using at least one step of immunopurification; c) detecting an acute kidney injury (AKI) marker in the exosome.

In one embodiment, the urine sample is obtained from the first urination in the morning for catheterized patients, and from the second urination for other patients and is maintained at −80° C. until it is analyzed.

The present invention considers as means for enriching the urine sample in exosomes at least one step of immunopurification. In particular, by using antibodies directed to the intracellular, extracellular or any domain of particular proteins that are preferably and differentially expressed in specific structures of the kidney, allows enrichment of exosomes. Thus, the method of the present invention considers antibodies directed to intracellular, extracellular or any domain of proteins that are preferably and differentially expressed in specific structures of the kidney, and suitable buffer to allow the antibodies to interact with the domains of proteins present in the external surface of exosomes.

In one embodiment, the method of the present invention can include at least one further mean for enrichment of exosomes, previous to the immunopurification.

The means for enriching the urine sample in exosomes also considered in the invention are laboratory methods and devices useful in separating larger elements from the urine sample, which could interfere in the detection phase later. For example, the interfering elements can be cells from the patient. Centrifuge tubes can be considered enriching means, since the processing in a laboratory centrifuge allows enrichment of exosomes, eliminating larger particles, such as for example cells. In one embodiment, the sample is centrifuged for 5 to 30 minutes at 5000 to 10000 rpm. In another embodiment, the sample is ultracentifuged for 30 to 120 minutes at 30000 to 45000 rpm.

Microfilter cartridges, microfilter columns, or other microfilter media, up to 0.22 micrometers are also considered as exosome enriching means, since microfiltration allows passing of exosomes through microfilters, thus enriching the urine sample in exosomes.

In another embodiment of the invention, the method considers, as means for detecting and/or quantifying a predetermined kidney injury marker, a primary antibody directed to a predetermined kidney injury marker, and a secondary antibody conjugated with a label, directed to the primary antibody. The label of the secondary antibody can be a fluorescent marker, an enzyme, a radioactive marker, a chemical compound, an infrared compound.

Optionally, the primary antibody can be conjugated directly with a label, in which case, the secondary antibody is not needed. The primary antibody can be also conjugated with a fluorescent marker, an enzyme, a radioactive marker, a chemical compound, an infrared compound.

In one embodiment, the immunopurification of a specific exosomal fraction from a specific kidney structure is carried out with an antibody selected, but not limited to: anti-aquoporin-1 (anti-AQP-1), anti-aquoporin-2 (anti-AQP-2), anti-aquoporin-3 (anti-AQP-3), anti-NKCC2, anti-NHE-3 and/or anti-NaPiII or a combination thereof.

In one embodiment, the specific kidney injury marker is selected, but not limited to NGAL, Cystatin-3, KIM-1, IL-1beta, and/or IL-18, or a combination thereof.

In a preferred embodiment, the immunopurification of a specific exosomal fraction from a specific kidney structure is carried out with antibodies directed to any domain of anti-AQP1, anti-AQP2, anti-AQP3, anti-NKCC2, anti-NHE-3, anti-NaPiII, or a combination thereof as means for enriching the urine sample in exosomes.

In a further preferred embodiment, the specific kidney injury marker is selected from NGAL, Cystatin-3, KIM-1, IL-1beta, IL-18 or a combination thereof.

In a more preferred embodiment, when anti-AQP-1 is used for immunopurifying a specific exosome fraction from a specific kidney structure, the kidney injury marker which presence and/or level is determined is NGAL and/or Cystatin-3, when anti-AQP-2 is used for immunopurifying a specific exosome fraction from a specific kidney structure, the kidney injury marker which presence and/or level is determined is NGAL, and when anti-AQP-3 is used for immunopurifying a specific exosome fraction from a specific kidney structure, the kidney injury marker which presence and/or level is determined is KIM-1, IL-1beta and/or Cystatin-3.

In one embodiment, when anti-AQP-1 is used for immunopurifying a specific fraction from a specific kidney structure, the exosomal fraction comes from the proximal tubule, and the presence and/or level of a specific marker indicates injury in said structure.

In one embodiment, when anti-AQP-2 is used for immunopurifying a specific fraction from a specific kidney structure, the exosomal fraction comes from the distal tubule, and the presence and/or level of a specific marker indicates injury in said structure.

In one embodiment, when anti-AQP-3 is used for immunopurifying a specific fraction from a specific kidney structure, the exosomal fraction comes from the collecting duct, and the presence and/or level of a specific marker indicates injury in said structure.

In one embodiment, when anti-NKCC2 is used for immunopurifying a specific fraction from a specific kidney structure, the exosomal fraction comes from the Henle's loop, and the presence and/or level of a specific marker indicates injury in said structure.

In one embodiment, when anti-NHE-3 is used for immunopurifying a specific fraction from a specific kidney structure, the exosomal fraction comes from the proximal tubule, and the presence and/or level of a specific marker indicates injury in said structure.

In one embodiment, when anti-NaPiII is used for immunopurifying a specific fraction from a specific kidney structure, the exosomal fraction comes from the proximal tubule, and the presence and/or level of a specific marker indicates injury in said structure.

The method optionally includes a step for evaluating the acute kidney injury in a subject for monitoring, diagnosis, prognosis and/or determining a treatment in the subject based on the presence and/or level of the specific kidney injury marker.

The invention further comprises a diagnostic kit for determining the presence and/or level of a specific kidney injury marker, for simple and early determination of the onset of AKI in a subject, the kit comprising means for enriching the urine sample in exosomes, using at least one step of immunopurification, and means for detecting a predetermined kidney injury marker of a condition.

Optionally, the diagnostic kit comprises means for obtaining a urine sample from a patient. In a particular embodiment, the means for obtaining the urine sample are selected among a urinary probe, in the case the patient is unable to provide a urine sample by him/her self; or a container to receive a urine sample from a patient.

The present invention considers as means for enriching the urine sample in exosomes at least one step of immunopurification. In particular, by using antibodies directed to the intracellular, extracellular or any domain of particular proteins that are preferably and differentially expressed in specific structures of the kidney, allows enrichment of exosomes. Thus, kit of the present invention considers antibodies directed to intracellular, extracellular or any domain of proteins that are preferably and differentially expressed in specific structures of the kidney, and suitable reaction buffer to allow the antibodies to interact with the domains of proteins present in the external surface of exosomes. The kit also comprises blocking agents or solutions and stock solutions of the specific kidney injury markers.

In one embodiment, the kit comprises a 96 well plate wherein the wells are covered with the antibodies directed to the intracellular, extracellular or any domain of the particular proteins that are preferably and differentially expressed in specific structures of the kidney, allowing enrichment of exosomes.

In one embodiment, the kit of the present invention can include at least one further mean for enrichment of exosomes, previous to the immunopurification.

The means for enriching the urine sample in exosomes considered in the invention are laboratory methods and devices useful in separating larger elements from the urine sample, which could interfere in the detection phase later. For example, the interfering elements can be cells from the patient. Centrifuge tubes can be considered enriching means, since the processing in a laboratory centrifuge allows enrichment of exosomes, eliminating larger particles, such as for example cells. Microfilter cartridges, microfilter columns, or other microfilter media, up to 0.22 micrometers are also considered as exosome enriching means, since microfiltration allows passing of exosomes through microfilters, thus enriching the urine sample in exosomes.

In another embodiment of the invention, the kit considers, as means for detecting and/or quantifying a predetermined kidney injury marker, a primary antibody directed to a predetermined kidney injury marker, and a secondary antibody conjugated with a label, directed to the primary antibody. The label of the secondary antibody can be a fluorescent marker, an enzyme, a radioactive marker, a chemical compound, an infrared compound.

Optionally, the primary antibody can be conjugated directly with a label, in which case, the secondary antibody is not needed. The primary antibody can be also conjugated with a fluorescent marker, an enzyme, a radioactive marker, a chemical compound, an infrared compound.

In one embodiment, the kit comprises antibodies selected, but not limited to: anti-aquoporin-1 (anti-AQP-1), anti-aquoporin-2 (anti-AQP-2), anti-aquoporin-3 (anti-AQP-3), anti-NKCC2, anti-NHE-3 and/or anti-NaPiII or a combination thereof.

In one embodiment, the kit comprises primary antibodies, either conjugated with a label or not for detection of a marker selected, but not limited to NGAL, Cystatin-3, KIM-1, IL-1beta, and/or IL-18 or a combination thereof.

In a preferred embodiment, the kit comprises antibodies directed to any domain of anti-AQP1, anti-AQP2, anti-AQP3, anti-NKCC2, anti-NHE-3, anti-NaPiII, or a combination thereof as means for enriching the urine sample in exosomes, when using immunopurification as a method for enrichment.

In a further preferred embodiment, the kit comprises primary antibodies, either conjugated with a label or not, for detection of NGAL, Cystatin-3, KIM-1, IL-1beta, IL-18.

In a more preferred embodiment, when anti-AQP-1 is used for immunopurifying a specific exosome fraction from a specific kidney structure, the kidney injury marker which presence and/or level is determined is NGAL and/or Cystatin-3, when anti-AQP-2 is used for immunopurifying a specific exosome fraction from a specific kidney structure, the kidney injury marker which presence and/or level is determined is NGAL, and when anti-AQP-3 is used for immunopurifying a specific exosome fraction from a specific kidney structure, the kidney injury marker which presence and/or level is determined is KIM-1, IL-1beta and/or Cystatin-3.

In one embodiment, when anti-AQP-1 is used for immunopurifying a specific fraction from a specific kidney structure, the exosomal fraction comes from the proximal tubule, and the presence and/or level of a specific marker indicates injury in said structure.

In one embodiment, when anti-AQP-2 is used for immunopurifying a specific fraction from a specific kidney structure, the exosomal fraction comes from the distal tubule, and the presence and/or level of a specific marker indicates injury in said structure.

In one embodiment, when anti-AQP-3 is used for immunopurifying a specific fraction from a specific kidney structure, the exosomal fraction comes from the collecting duct, and the presence and/or level of a specific marker indicates injury in said structure.

In one embodiment, when anti-NKCC2 is used for immunopurifying a specific fraction from a specific kidney structure, the exosomal fraction comes from the Henle's loop, and the presence and/or level of a specific marker indicates injury in said structure.

In one embodiment, when anti-NHE-3 is used for immunopurifying a specific fraction from a specific kidney structure, the exosomal fraction comes from the proximal tubule, and the presence and/or level of a specific marker indicates injury in said structure.

In one embodiment, when anti-NaPiII is used for immunopurifying a specific fraction from a specific kidney structure, the exosomal fraction comes from the proximal tubule, and the presence and/or level of a specific marker indicates injury in said structure.

The kit of the invention optionally includes instructions for using the kit.

The method and the kit of the invention comprise a step of immunopurification of urinary exosomes. Therefore, the invention also describes a method for inmunopurifying urinary exosomes and the use of antibodies for purifying urinary exosomes.

The invention describes a method for purifying urinary exosomes, the method comprising the steps of (a) incubating a urine sample, or optionally, a decellularized urine sample with an antibody directed to the intracellular, extracellular, or any domain of proteins preferably and differentially expressed in the surface of different kidney structures, thus forming an exosome-antibody complex; (b) incubating the exosome-antibody complex resulting from (a) with a tag that recognizes any region of the antibody and is bound to an insoluble agent, thus forming an exosome-antibody-tag-insoluble agent complex; (c) separating the exosome-antibody-tag-insoluble agent complex from the supernatant; (d) washing of the exosome-antibody-tag-insoluble agent complex with an adequate buffer.

In one embodiment the urine sample, or decellularized urine sample is incubated from 20 to 60 minutes with the antibody, in an adequate buffer, at room temperature.

In one embodiment, the antibodies used in the purification method for urinary exosomes are selected, but not limited to: anti-aquoporin-1 (anti-AQP-1), anti-aquoporin-2 (anti-AQP-2), anti-aquoporin-3 (anti-AQP-3), anti-NKCC2, anti-NHE-3 and/or anti-NaPiII or a combination thereof.

In a preferred embodiment, the antibodies used in the purification method for urinary exosomes are directed to any domain of anti-AQP1, anti-AQP2, anti-AQP3, anti-NKCC2, anti-NHE-3, anti-NaPiII, or a combination thereof.

In one embodiment, when anti-AQP-1 is used for immunopurifying a specific fraction from a specific kidney structure, the exosomal fraction comes from the proximal tubule, and the presence and/or level of a specific marker indicates injury in said structure.

In one embodiment, when anti-AQP-2 is used for immunopurifying a specific fraction from a specific kidney structure, the exosomal fraction comes from the distal tubule, and the presence and/or level of a specific marker indicates injury in said structure.

In one embodiment, when anti-AQP-3 is used for immunopurifying a specific fraction from a specific kidney structure, the exosomal fraction comes from the collecting duct, and the presence and/or level of a specific marker indicates injury in said structure.

In one embodiment, when anti-NKCC2 is used for immunopurifying a specific fraction from a specific kidney structure, the exosomal fraction comes from the Henle's loop, and the presence and/or level of a specific marker indicates injury in said structure.

In one embodiment, when anti-NHE-3 is used for immunopurifying a specific fraction from a specific kidney structure, the exosomal fraction comes from the proximal tubule, and the presence and/or level of a specific marker indicates injury in said structure.

In one embodiment, when anti-NaPiII is used for immunopurifying a specific fraction from a specific kidney structure, the exosomal fraction comes from the proximal tubule, and the presence and/or level of a specific marker indicates injury in said structure.

In one embodiment, the tag that recognizes any region of the antibody is protein A or protein G or fractions thereof, bound to agarose or sepharose beads.

In another embodiment, the tag is biotin or a fraction thereof, and is bound to the antibody. Thus, the complex exosome-antibody-biotin is formed. In this embodiment, the insoluble agent are magnetic beads bound to a compound with affinity to biotin or a fraction thereof, such as, but not limited to an antibody. Thus, adding the magnetic beads, the complex exosome-antibody-biotin-compound with affinity to biotin-magnetic bead is formed and then separated in the next step.

In one embodiment, the separation of the exosome-antibody-tag-insoluble agent from the supernatant is performed by centrifugation or sedimentation.

The invention also discloses the use of an antibody or a combination of antibodies directed to the intracellular, extracellular, or any domain of proteins preferably and differentially expressed in the surface of different kidney structures for immunopurification of urinary exosomes.

In one embodiment, the antibodies used for immunopurification of urinary exosomes are selected, but not limited to: anti-aquoporin-1 (anti-AQP-1), anti-aquoporin-2 (anti-AQP-2), anti-aquoporin-3 (anti-AQP-3), anti-NKCC2, anti-NHE-3 and/or anti-NaPiII or a combination thereof.

In a preferred embodiment, the antibodies used for immunopurification of urinary exosomes are directed to any domain of anti-AQP1, anti-AQP2, anti-AQP3, anti-NKCC2, anti-NHE-3, anti-NaPiII, or a combination thereof.

In one embodiment, when anti-AQP-1 is used for immunopurification of urinary exosomes a specific fraction from a specific kidney structure, the exosomal fraction comes from the proximal tubule, and the presence and/or level of a specific marker indicates injury in said structure.

In one embodiment, when anti-AQP-2 is used for immunopurification of urinary exosomes a specific fraction from a specific kidney structure, the exosomal fraction comes from the distal tubule, and the presence and/or level of a specific marker indicates injury in said structure.

In one embodiment, when anti-AQP-3 is used for immunopurification of urinary exosomes a specific fraction from a specific kidney structure, the exosomal fraction comes from the collecting duct, and the presence and/or level of a specific marker indicates injury in said structure.

In one embodiment, when anti-NKCC2 is used for immunopurification of urinary exosomes a specific fraction from a specific kidney structure, the exosomal fraction comes from the Henle's loop, and the presence and/or level of a specific marker indicates injury in said structure.

In one embodiment, when anti-NHE-3 is used for immunopurification of urinary exosomes a specific fraction from a specific kidney structure, the exosomal fraction comes from the proximal tubule, and the presence and/or level of a specific marker indicates injury in said structure.

In one embodiment, when anti-NaPiII is used for immunopurification of urinary exosomes a specific fraction from a specific kidney structure, the exosomal fraction comes from the proximal tubule, and the presence and/or level of a specific marker indicates injury in said structure.

The method and kit of the invention have the advantage to provide a specific, simple and early detection of kidney injury markers that is no possible with the standard methods applied until now. The method and kit of the invention also provide the advantage of identifying the specific structure that is injured in the kidney, since it uses specific antibodies for specific proteins present in specific structures of the kidney.

The method and kit of the present invention can be used for monitoring, diagnosis, prognosis and/or determination of treatment in any subject. Particularly, the method and kit of the invention is useful for monitoring the development of renal injury in patients that underwent kidney transplant, due to the ischemia-reperfusion process associated with the transplant. The method and kit of the invention is also useful for monitoring, diagnosis, prognosis and/or determination of treatment in patients in intensive care unit (ICU) that could develop AKI and need an early detection.

### DESCRIPTION OF THE FIGURES

FIG. 1: Representative Western blot comparing the abundance of NGAL in the cellular fraction and exosomal fraction from a urine sample of a kidney transplant patient at post-transplant days 1, 2, 3, and 4.

FIG. 2: Representative Western blot showing abundance of NGAL-24 and NGAL-46 in complete urine (U) and exosomal fraction (E) from a patient on day 1 post-transplant.

FIG. 3: Graphical representation of NGAL in the total exosomal fraction of urine in patients day 1 post-transplant. AU=Arbitrary units.

FIG. 4: Relationship between NGAL-46 and AQPs 1, 2, and 3 (* p<0.05, n=5), AU=Arbitrary units.

FIG. 5: Comparative analysis between two levels of SCR and NGAL in individuals with kidney transplant.

FIG. 6: Relative abundance of NGAL (Arbitrary Units) in exosome-free urine (white bars, EF-U) and total exosome fraction (black bars, TE) in ICU patients classified as AKI1 (5 patients), AKI2 (3 patients), and AKI 3 (5 patients).

FIG. 7: TE/EF-U ratio of NGAL abundance in arbitrary units in patients from ICU classified as AKI1 (5 patients), AKI2 (3 patients) and AKI3 (5 patients).

FIG. 8: Relative NGAL abundance in exosome-free urine (EF-U), total exosomes (TE) and immunopurified exosomes (IP-E) in ICU patients classified as AKI1 (5 patients).

### EXPERIMENTAL SECTION

In this section illustrative examples are given as guidance, therefore, these examples are in no way to be construed as limiting

### EXAMPLES OF APPLICATION

Some of the embodiments of the present invention are described below, based on a study determining the renal or kidney injury in a group of 12 human patients that underwent a kidney transplant and a study comprised 28 individuals admitted to the ICU, where 46% (13/28) developed AKI according to RIFLE classification.

### Example 1

### Description of the Group Under Study

The method and products of the present invention were tested by analysing a group of 12 human patients, plus healthy individuals acting as a control group. The inclusion criteria used for healthy controls were the absence of renal symptomatology and the inclusion criteria for patients were all individuals who underwent kidney transplant during the period of duration of the present study. Patients were enrolled considering an informed consent approval.

### Example 2

### Urine Sampling

50 ml of urine catheterized specimen was obtained from each individual under study. The sample was taken from the first urination in the morning, by a trained nurse. In the case of transplanted individuals, samples were obtained the day previous to the transplant and three samples post-surgery in the following three days, once a day, of which, the first sample post-surgery was chosen for analysis. Samples were maintained at −80° C. waiting for Western Blot analysis.

### Example 3

### Preparation of Urinary Exosomes

Exosomes were obtained considering two different approaches. The first one, corresponded to a centrifuge separation in two steps, the first using centrifugation, and the second step using ultracentrifugation. The second method, the main feature of the present invention, considered a first stage of centrifugation, and a second step of immunopurification.

In the first method, with 2 centrifugation steps, 10 ml of the urine sample were taken, to which a protease inhibitor tablet was added (Complete Mini protease inhibitor cocktail tablets, Roche). A first centrifugation was performed at 17,000 g for 15 minutes at 4° C. for separating the cellular content, which was stored at −80° C., obtaining a first supernatant fraction (S1). A first fraction of Si was further ultracentrifuged at 38,000 rpm for 1 hour, which originated a second supernatant fraction (S2) and a precipitate (total exosomes).

In the case of a first step of centrifugation, with a second step considering immunopurification, a fraction of S1, obtained as described in the first step for the method with 2 centrifugation steps, was used for immunopurification of exosomes using AQP1, AQP2, or AQP3 antibodies for obtaining exosomes from different regions of the kidney. Samples were resuspended in a load buffer (100 mM Tris-HCl, pH 6.8, 200 mM dithiotreitol (DTT), 4% SDS, 0.2% bromophenol blue, 20% glycerin), so as to load 100 micrograms of total proteins of each sample in a gel.

### Example 4

### Western Blot Analysis

Western blot allowed identification of the presence of proteins using specific antibodies. Proteins were separated according to their size using electrophoresis in 7.5% and 12% acrylamide gels (SDS-PAGE) for 90 minutes at 100 V. Once separated, all proteins were transferred to nitrocellulose membranes for 90 minutes at 300 mA. After this process, the membranes were blocked using 5% skim milk in constant agitation for 1 hour, for later incubation with a primary anti-AQP1 (Santa Cruz Biotechnology), anti-AQP2 (Santa Cruz Biotechnology), anti-AQP3 (Santa Cruz Biotechnology), or anti-NGAL (R&D Systems) primary antibodies, overnight at 4° C. with agitation. Once the incubation ended, the membranes were washed 3 times with 1% PBS-Tween 20 (Winkler), to proceed with a secondary antibody for 2 hours at room temperature with agitation. Finally, the membranes were washed 3 times with PBS-Tween 20 1% for further processing.

Abundance of proteins was determined by a semi-quantitative analysis, using densitometric determination of bands obtained by Western Blot, using Adobe Photoshop CS4 software, with 2 measurements (total band and a fixed size band for all samples) for each band obtained as to standardize the determination.

Protein Determination

Total protein concentration was determined in both cellular fractions, using a commercially available kit (BCA Protein Assay Reagent, Pierce).

Statistical Analysis

A descriptive and associative study were conducted using the statistical model ANOVA. The analysis was performed using WINKS SDA 6.0 software, considering as significant differences with a p-value<=0.05.

### Example 5

### Results of the Analysis According to the Method of the Present Invention

Characteristics of the Group Study

12 patients that received kidney transplant were considered in the present study.

Ages of patients ranged from 15 to 60 years, while most of the patients (7 out of 12) were between 40 to 60 years. Most of the patients were male (91.6%). 25% received an organ from a cadaver donor, and 75% from a living donor (Table 4).

Serum creatinine is an important parameter to test the viability of the transplanted organ. The first day after transplant, all of the patients had values for serum creatinine higher than normal (0.8 to 1.4 mg/dl), showing higher values the patients whom received the organ from a cadaver donor (Table 4).

Other parameter considered was the ischemic time to which the organ was subjected to, previous to the transplant. The times range from 27 to 1182 minutes, being in general the higher times for those patients whom received the organ form a cadaver donor, with times ranging from 300 to 1182 minutes, compared to the times from patients receiving the organ from a living donor, from 27 to 360 minutes (Table 4).

TABLE 4
Patient information in the study

| Patient | Age | | | SCR day + 1 | Ischemic time |
|---|---|---|---|---|---|
| N^{o} | (years) | Sex | Donor type | (mg/dl) | (minutes) |
| 1 | 60 | M | CD | 12.5 | 1182 |
| 2 | 57 | M | LD | 7.4 | 27 |
| 3 | 16 | M | CD | 6.1 | 300 |
| 4 | 34 | M | LD | 4.8 | 120 |
| 5 | 15 | F | LD | 2.3 | N/A |
| 6 | 27 | M | LD | 4.7 | 90 |
| 7 | 42 | M | LD | 9.3 | 360 |
| 8 | 46 | M | LD | 2.7 | N/A |
| 9 | 46 | M | LD | 6.1 | N/A |
| 10 | 55 | M | LD | 5.5 | 120 |
| 11 | 28 | M | LD | 2.8 | 60 |
| 12 | 42 | M | CD | 9.0 | 720 |

Sex: M = Male, F = Female; Donor type CD = Cadaver donor, LD = Living donor; SCR day + 1 = Serum creatinine 1 day post-transplant; Ischemic time: N/A = No data available.

Analysis of Renal Failure Biomarkers

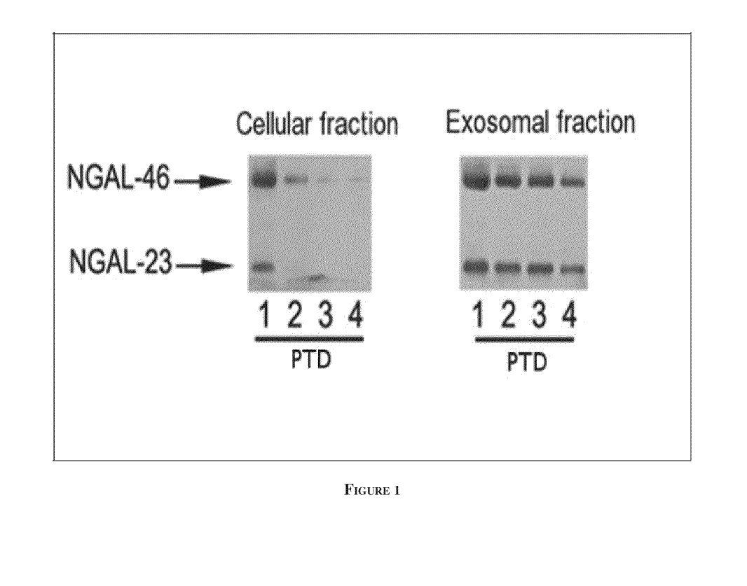

Previous results obtained by our research team, and the results from the present study, show that in healthy individuals there are no detectable levels of biomarkers in the cell fraction or in the exosomal fraction of urine (Flores M., Marquez S. 2009, Research unit: New molecular markers for the study of kidney transplant dysfunction, Universidad de los Andes, Santiago, Chile). Also, biomarker expression levels in exosomal fraction are higher than those expressed in the cell fraction (FIG. 1). Furthermore, higher levels of expression of biomarkers under study occur the first day post-transplant (FIG. 1). Finally, using Western blot it is possible to detect two NGAL isoforms, NGAL-23 of 23 kDa and NGAL-46 of 46 kDa (FIG. 1). Thus, the present study is focused in determining the profile of biomarkers from the exosomal fraction of a urine sample collected the first day post-transplant.

NGAL

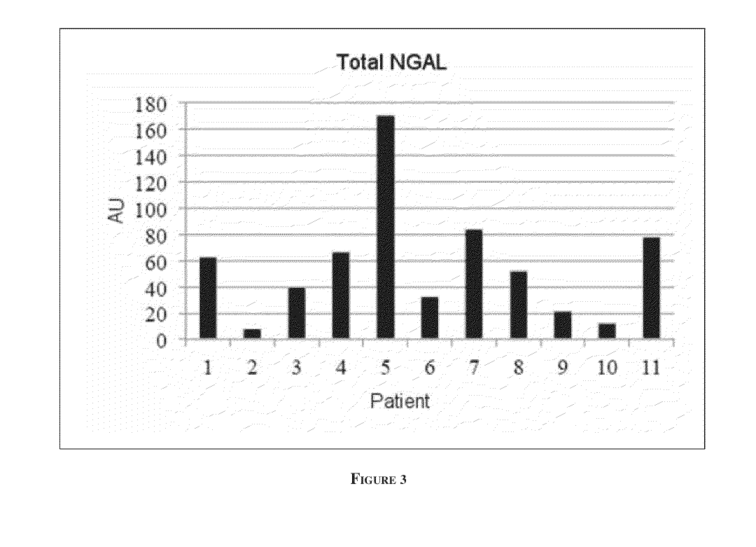

All NGAL isoform abundance was analysed (NGAL-23 and NGAL-46) from total urinary exosomes from the first day post-transplant, using Western blot techniques, in 12 patients included in the study, obtaining information for 11 of them. These results show that NGAL was expressed in almost all of the cases. The range of expression was between 7.3 and 170.3 AU, i.e. about 20 times of difference between the extreme cases (FIG. 3).

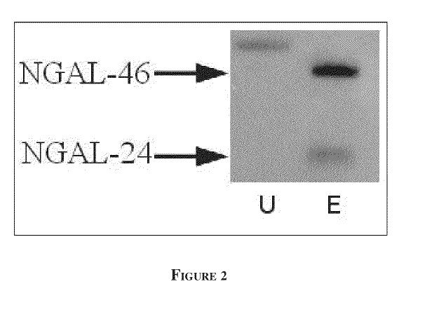

Using Western blot to identify NGAL-46 and NGAL-24 both markers are expressed only in urinary exosomal fraction, while the supernatant only shows a non-specific band (FIG. 2).

From 11 patients, those with higher NGAL values were patients 5 and 7, with values 170.3 and 83.8 AU, respectively.

The lowest level was found for patient 10 with a value of 12.6 AU and patient 2, with 7.9 AU.

Expression of Biomarkers in Immunopurified Exosomes

Pisitkun in 2004 (Pisitkun T., Shen R. F., Knepper M. A. 2004, Proc Natl Acad Sci USA, 101:13368-13373) described the presence of aquaporins (AQPs) in urinary exosomes from normal patients. Thus, the present study looked for AQPs (AQP1, AQP2, AQP3) expression in immunopurified exosomes from patients whom underwent renal transplant. The election of each of these components is because its expression is associated with specific zones in the nephron. AQP1 is usually associated with proximal tubules (Knepper M. A. 1997, Am J Physiol, 272:F3-F12), AQP2 is associated with distal tubules and collecting ducts (Ma T., Yang B., Gillespie A., Carlson E. J., Epstein C. J., Verkman A. S. 1998, J Biol Chem, 273:4296-4299), and AQP3 is associated with collecting ducts from the nephron (Sasaki S., Ishibashi K., Marumo F. 1998, Annu Rev Physiol, 60:199-220).

This analysis allows determining the abundance of each biomarker present in a particular type of exosome. Thus, the approach is to establish a relationship between the potential origin of the biomarker of renal injury analysed.

NGAL-46

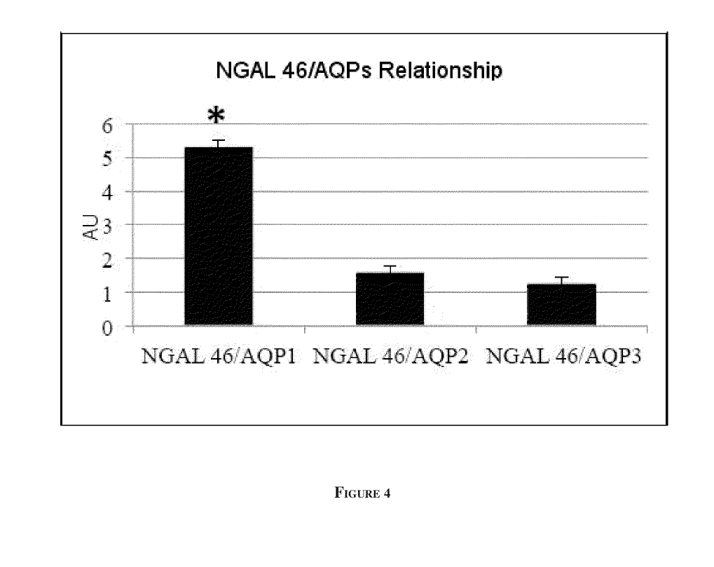

FIG. 4 shows the normalization of NGAL-46 isoform by the level of AQPs in urinary exosomes of patients with positive information. NGAL-46 was chosen since the expression of NGAL-46 is higher than NGAL-23 (FIG. 1).

The results show that NGAL abundance in relationship to the abundance of AQP1 is statistically higher than other AQPs (FIG. 4, p<0.05). These results suggest that exosomes containing AQP1 has a higher abundance of NGAL (FIG. 4). Based on these results, it shows that NGAL-46 is mainly associated with regions of the kidney where AQP1 is preferentially expressed, i.e. the proximal zone of the collecting duct.

Comparison of Serum Creatinine Levels with NGAL

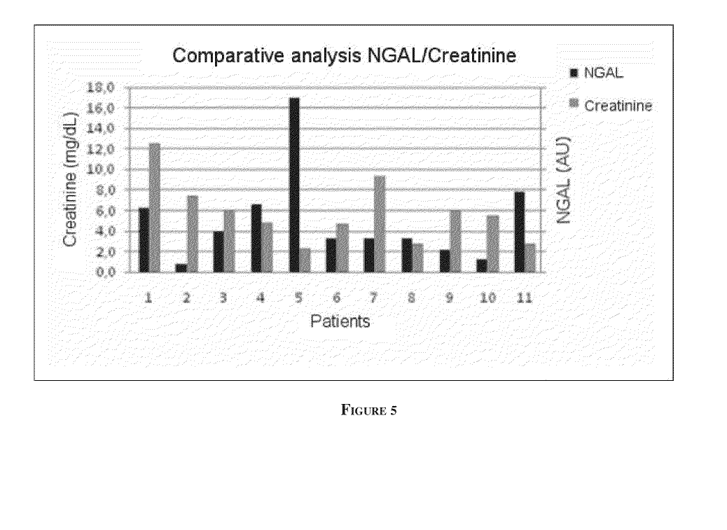

Results comparing the levels of NGAL and serum creatinine (SCR) show that individuals with SCR levels lower than 3 mg/dl (patients 5, 8, and 11) o higher than 6 mg/dl (patients 1, 2, 3, 7, 9, and 12), do not have a correlation with NGAL (FIG. 5).

The data suggests that markers of glomerular filtration (SCR) are not correlated with markers of renal injury (NGAL).

Discussion

Analysis of Renal Injury Biomarkers

NGAL

Flores and Marquez (2009) (Flores M., Marquez S. 2009, Research unit: New molecular markers for the study of kidney transplant dysfunction, Universidad de los Andes, Santiago, Chile) showed that there are no detectable levels of NGAL in the exosomal fraction of urine from healthy individuals. 11 of patients of the present study for which information was available showed that NGAL was detected in 100% of the urinary exosomes of patients that underwent renal transplant but it was not detected in the supernatant free of the cell fraction of urine. Levels of expression are between 7.9 and 170.3 AU, with over 22 times between the extreme individuals. Thus, the data suggests that NGAL detection in urinary exosomes constitutes an excellent biomarker for renal injury, since the analysed fraction was more representative than complete urine.

Relationship of NGAL with the Type of Donor

It is known that the quality of the transplanted organ is better when the donor is alive compared to a cadaver donor, which should be reflected as a lower expression of renal injury markers. This type of behavior was shown in patients 2, 6, 9, and 10, with the lower levels of NGAL and also received the transplanted organ from a living donor. In the same context patient 1 shows a higher level of NGAL, which can be explained since this patient received a transplant from a cadaver donor. Nevertheless, this pattern of expression of NGAL is not shown by patient 3, which received a transplant from a cadaver donor and shows moderate levels of NGAL, and thus could be interpreted as the quality of the organ.

Patients 5 and 7 show higher levels of NGAL although they received a kidney from a living donor. Patient 7 showed a delayed function of transplanted kidney, requiring hemodialysis during the first week post-transplant. Thus, NGAL is an excellent marker for renal injury.

Relationship of NGAL with Ischemic Time

Ischemic time of an organ is a determinant factor of the damage the organ suffers before transplant, since the longer this time, the lower the function it will have. This is observed in patients 1 and 7, which have a high expression of NGAL related with ischemic values of 360 and 1182 minutes. The contrary is observed in patients 2 and 6, with ischemic values of 27 and 90 minutes. This relationship can be established in most of the cases, with exception of patient 11, with a low value of ischemic time and high levels of NGAL (Table 4, FIG. 3). Patient 5 shows the higher level of NGAL of the group, but there is no available data of ischemic time.

Relationship of NGAL with Age and Sex

According to the results obtained in the present study, age seems to have no relationship with NGAL levels for each patient, since, for example patient 5 shows the highest levels of NGAL (FIG. 3) even though is the youngest person in the study. It is not possible either to establish a relationship between sex of the patient and the marker, since the study only included one woman.

Expression of Biomarkers in Immunopurified Exosomes

AQP1, AQP2, and AQP3

Abundance of biomarkers was analysed in exosomes immunopurified with different types of AQPs.

Knepper 1997 (Knepper M. A. 1997, Am J Physiol, 272:F3-F12), showed that AQP1 is preferentially associated with proximal tubules, while AQP2 is associated with distal tubules and collecting ducts (Ma T., Yang B., Gillespie A., Carlson E. J., Epstein C. J., Verkman A. S. 1998, J Biol Chem, 273:4296-4299), and AQP3 is associated with collecting ducts from the nephron (Sasaki S., Ishibashi K., Marumo F. 1998, Annu Rev Physiol, 60:199-220).

Pisitkun in 2004 (Pisitkun T., Shen R. F., Knepper M. A. 2004, Proc Natl Acad Sci USA, 101:13368-13373) showed that AQPs were present in urinary exosomes of normal patients.

The present study shows that, AQPs, besides being present in urinary exosomes from normal patients are also present in all patients subjected to renal transplant, independently of the condition of the organ or the donor at the moment of transplant (Table 4).

Relationship Between Membrane Components and Biomarkers

Considering that AQP1 is associated with proximal tubules in nephrons ((Knepper M. A. 1997, Am J Physiol, 272:F3-F12), and AQP2 is associated with distal tubules, and AQP3 is associated with collecting ducts, the abundance of NGAL was analysed in exosomes immunopurified with AQP1, AQP2, and AQP3.

NGAL

Expression of NGAL in each of the immunopurified exosomes suggests a significantly higher abundance of NGAL in exosomes with AQP1 in their membranes (p<0.05, FIG. 4), followed by exosomes with AQP2 and AQP3, with no significant difference among the latter two. The data suggests that exosomes come from the proximal tubule of the kidney have a higher abundance of renal injury marker NGAL.

Conclusions

Analysis of biomarkers in renal injury at the first day post-transplant allowed detection of biomarkers using Western blot techniques to the exosomal fraction of urine, but not full urine. These biomarkers are not detectable using this technique in healthy patients.

NGAL is present in the exosomal fraction of urine samples taken the first day post-transplant of the kidney in all patients analysed, suggesting that the expression of molecular factors in the exosomes is due to development of renal injury in the transplant, associated to the transplant procedure. Interestingly, patients 1, 4, and 7 showed together high levels of NGAL. Of these patients, 1 and 7 showed a retarded function of the transplant, requiring dialysis therapy during the first week after transplant. These preliminary results, show that increased levels of NGAL in urinary exosomes play a clinical role in establishing renal dysfunction in renal transplants.

Using quantitative methods for determination of the abundance of these markers, for example using ELISA, allows establishing limits for determining the degree of renal injury associated with the renal transplant and their implications in function and viability of the transplant.

Based on the analysis, NGAL is mainly expressed in urinary exosomes obtained from proximal tubules.

### Example 6

### Description of the Group Under Study

The same procedures used in previous examples were used in terms of statistical analysis, sample preparation, Western blotting protocols, total urine, total exosomes, immunopurified exosomes.

In this Example, 30 ml urine samples were obtained from the second urination and kept in a sterile container, from patients admitted at the Intensive Care Unit (ICU). The samples were kept at −80° C. until further processing and Western blot analysis.

Study Group

The study group comprised 28 individuals admitted to the ICU, with ages ranging from 21 to 95 years, 19 male and 9 female. 46% (13/28) developed AKI according to RIFLE classification.

In the Table 5 below are shown the age, sex, and AKI stage determined according to usual methods.

TABLE 5
Patient information in the study.

| Patient N^{o} | Age (Years) | Sex | AKI stage |
|---|---|---|---|
| 1 | 64 | M | AKI3 |
| 2 | 75 | M | AKI3 |
| 3 | 34 | M | N |
| 4 | 59 | F | N |
| 5 | 95 | M | AKI1 |
| 6 | 82 | F | N |
| 7 | 23 | M | AKI3 |
| 8 | 21 | M | AKI3 |
| 9 | 74 | M | AKI1 |
| 10 | 79 | F | AKI2 |
| 11 | 87 | F | N |
| 12 | 34 | M | N |
| 13 | 40 | F | N |
| 14 | 76 | M | N |
| 15 | 27 | M | N |
| 16 | 39 | M | AKI2 |
| 17 | 57 | F | N |
| 18 | 82 | M | N |
| 19 | 37 | M | N |
| 20 | 55 | F | N |
| 21 | 54 | F | N |
| 22 | 41 | M | AKI1 |
| 23 | 44 | F | AKI1 |
| 24 | 22 | M | AKI2 |
| 25 | 48 | M | AKI1 |
| 26 | 38 | M | N |
| 27 | 85 | M | AKI1 |
| 28 | 86 | M | N |

M: Male, F: Female, N: No AKI

46% of the 28 patients were diagnosed with AKI, 9 were women (32%) and 19 men (68%). Classification according to AKI stage shows that a higher number of men are the main constituent of the group.

57% (16 patients) were older than 45 years, although no relationship is observed in terms of AKI stage and age.

### Example 7

### Analysis of Biomarkers of Kidney Injury in Total Exosomes, Exosome-Free Urine, and Immunopurified Exosomes

AKI biomarker NGAL was analysed by Western blot techniques in Exosome-Free urine (EF-U), total exosome fraction (TE) and immunopurified exosomes (IP-E). From 28 patients in the study, 17 showed detectable levels of NGAL using Western blot technique. The Table 6 below shows the results for the detection of biomarkers in the patients of the study.

TABLE 6
Detection of biomarkers in the patients.

| Patient N^{o} | NGAL | AKI stage |
|---|---|---|
| 1 | + | AKI3 |
| 2 | + | AKI3 |
| 3 | − | N |
| 4 | + | N |
| 5 | + | AKI1 |
| 6 | + | N |
| 7 | + | AKI3 |
| 8 | + | AKI3 |
| 9 | + | AKI1 |
| 10 | + | AKI2 |
| 11 | − | N |
| 12 | + | N |
| 13 | − | N |
| 14 | − | N |
| 15 | − | N |
| 16 | + | AKI2 |
| 17 | + | N |
| 18 | − | N |
| 19 | − | N |
| 20 | + | N |
| 21 | − | N |
| 22 | + | AKI1 |
| 23 | + | AKI1 |
| 24 | + | AKI2 |
| 25 | + | AKI1 |
| 26 | − | N |
| 27 | + | AKI1 |
| 28 | − | N |

+: Presence of biomarker, −: absence of biomarker

100% (13/13) of the patients diagnosed with AKI (13/28) has detectable NGAL in at least one of the studied fractions. In the case of patients with negative diagnose for AKI, 33% show positive NGAL.

Furthermore, in patients classified with AKI, NGAL abundance was higher in the exosomal fraction in 85% of cases, compared to EF-U fraction. 5 patients, with AKI diagnose, showed preferential expression of NGAL in the exosomal fraction (patients 5, 8, 9, 12, and 16), while their levels of NGAL in EF-U fraction were extremely low. Only 2 patients with positive AKI diagnose and NGAL showed equivalent abundance of NGAL in both fractions (patients 2 and 7).

In the group with a negative AKI diagnose, 60% show undetectable levels of NGAL. All patients classified with negative AKI diagnose and positive NGAL, showed higher levels of NGAL in the exosomal fraction.

TABLE 7
Relative abundance of NGAL in analyzed
urinary fractions in the patients.

| Patient N^{o} | NGAL EF-U | Total Exosomes | AKI stage |
|---|---|---|---|
| 1 | + | ++ | AKI3 |
| 2 | ++ | ++ | AKI3 |
| 3 | − | − | N |
| 4 | + | ++ | N |
| 5 | − | + | AKI1 |
| 6 | + | ++ | N |
| 7 | ++ | ++ | AKI3 |
| 8 | − | + | AKI3 |
| 9 | − | + | AKI1 |
| 10 | + | ++ | AKI2 |
| 11 | − | − | N |
| 12 | − | ++ | N |
| 13 | − | − | N |
| 14 | − | − | N |
| 15 | − | − | N |
| 16 | − | + | AKI2 |
| 17 | − | + | N |
| 18 | − | − | N |
| 19 | − | − | N |
| 20 | − | + | N |
| 21 | − | − | N |
| 22 | + | ++ | AKI1 |
| 23 | + | ++ | AKI1 |
| 24 | + | ++ | AKI2 |
| 25 | + | ++ | AKI1 |
| 26 | − | − | N |
| 27 | + | ++ | AKI1 |
| 28 | − | − | N |

+: Presence of NGAL, −:Absence of NGAL

Patients were classified in three stages of AKI, being AKI3 the most serious. From 13 patients diagnosed with AKI, 5 were classified as AKI1 and the 5 (100%) showed higher abundance of NGAL in the exosomal fraction. From the 3 patients diagnosed with AKI2, 3 showed higher NGAL abundance in the exosomal fraction. From the 5 patients diagnosed with AKI3, 3 (60%) showed higher abundance of NGAL in the exosomal fraction (Table 7).

Furthermore, the relative abundance of NGAL between Exosome-free urine (EF-U) and total exosome (TE) fraction was analysed.

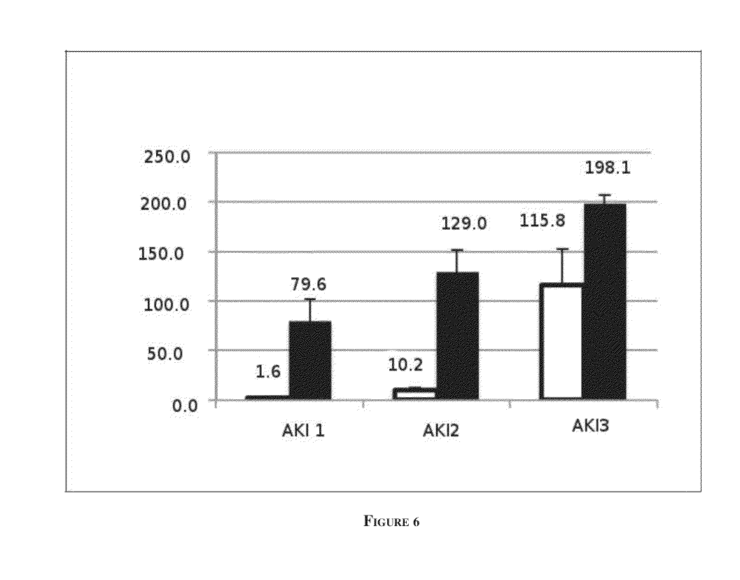

NGAL levels in the urine of patients admitted to the ICU and diagnosed with AKI1, AKI2, and AKI3 were determined separately. Results clearly show that with an increasing level of injury, NGAL abundance also increases, although NGAL abundance is higher in TE compared to EF-U in the case of AKI1 and AKI2 (FIG. 6).

In the group of patients with a negative AKI diagnose, 33% (5/15) showed detectable levels of NGAL in the TE fraction. NGAL abundance levels in this fraction was in average 13.5±9.8 AU. These levels were significantly lower than the ones found in TE of patients with AKI1 (79.6±22 AU, FIG. 6). This could be interpreted as an incipient kidney injury which was not detected by serum creatinine levels.

Previous data shows that measuring NGAL in TE is a method with a higher sensibility for AKI diagnose 24 hours before AKI diagnose using serum creatinine.

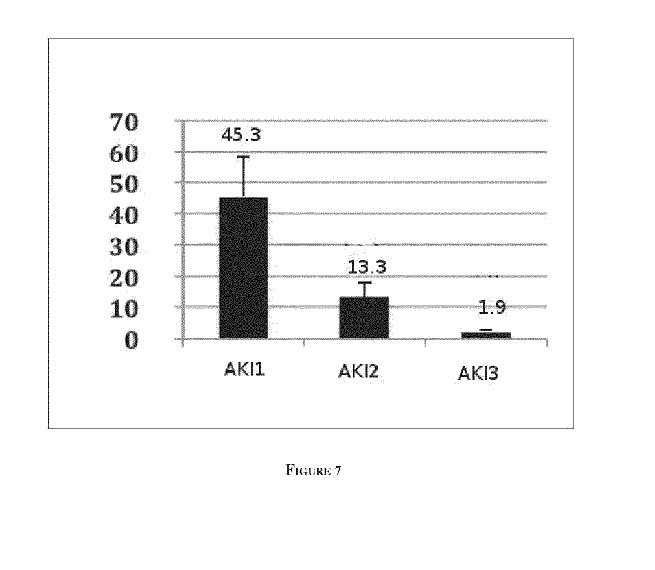

To compare NGAL relative abundance in TE and EF-U fractions, the ratio TE/EF-U was calculated. Results (FIG. 7) show that NGAL abundance is 45 times higher in TE compared to EF-U in the case of AKI1, 13 times higher in the case of AKI2. Nevertheless, patients diagnosed with AKI3 showed no significant difference. The data shows that NGAL in urinary exosomes has a high sensitivity in early detection of AKI (AKI1, and AKI2) in a period of 24 hours before establishing the diagnose of AKI using the serum creatinine method.

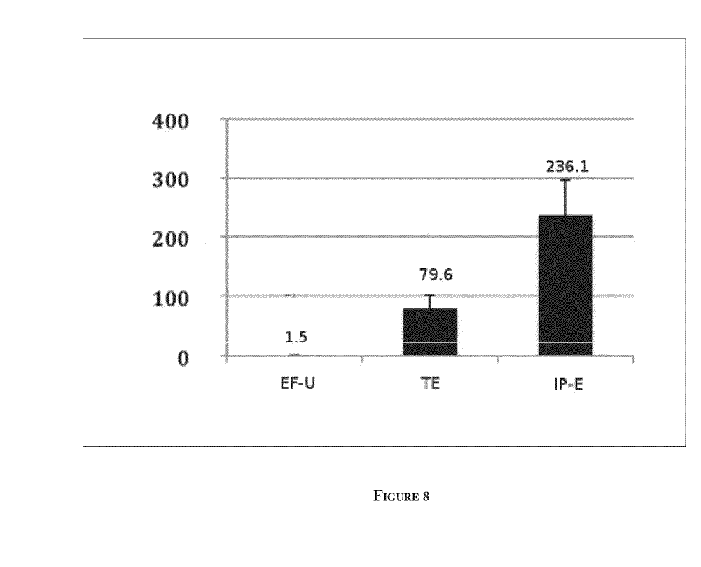

Since previous results show a higher abundance of NGAL in total exosomes (TE) in patients with AKI1, the abundance of NGAL in immunopurified exosomes (IP-E) was further analysed. AQP1 was used to immunopurify the exosomes in patients diagnosed with AKI1. The abundance of NGAL in IP-E fraction was compared to TE and EF-U fractions. The results show that NGAL abundance in IP-E is higher than TE and even higher to EF-U fractions (FIG. 8).

The data shows that immunopurifying exosomes from the proximal tubule section of the nephron increases the abundance of NGAL and thus the sensitivity in early detection of AKI in critical patients.

The analysis to the group of 28 patients of this example allows establishing the determination of NGAL in urine is a good method for AKI diagnosis, in a period of 24 hours before the usual method of creatinine levels in serum allows.

NGAL abundance in the exosomal fraction is higher than in exosome-free urine fraction, thus showing a higher sensitivity method for early AKI diagnose, in particular for stages AKI1 and AKI2.

Immunopurification of exosomes allows increasing the sensitivity of AKI detection due to higher abundance of NGAL in immunopurified exosomes with AQP1.

Given that other pathologies are able to increase levels of NGAL in urine, the analysis of immunopurified exosomes allows establishing that the measured NGAL comes from the kidney, increasing the specificity in determining exosomal biomarkers for early AKI detection.

1. Method for monitoring, diagnosis and/or prognosis of acute kidney injury in early stage and determination of treatment in subjects suffering thereof, the method comprising the following steps:
(a) providing a urine sample;
(b) enriching the urine sample in exosomes present in the urine sample using at least one step of immunopurification;
(c) detecting and/or quantifying an acute kidney injury (AKI) marker in the exosomes.

2-33. (canceled)
